(19) United States
(12) Patent Application Publication
(10) Pub. No.: US 2006/0013786 A1
(43) Pub. Date: Jan. 19, 2006

(54) Skin preparation for external use

(75) Inventor: Akinori Hanano, Hyogo (JP)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Noevir Co., LTD, Hyogo (JP)

(21) Appl. No.: 10/520,037

(22) PCT Filed: Jan. 9, 2003
(86) PCT No.: PCT/JP03/00101

§ 371 Date: Jun. 30, 2005

(30) Foreign Application Priority Data
Jul. 2, 2002 (JP) 2002-193944

(51) Int. Cl. A61K 8/86; A61K 8/72; A61K 31/19

(52) U.S. Cl. 424/70.11; 514/557

(57) ABSTRACT

An external preparation for skin is provided which can be readily applied to the skin surface in a uniform manner, is excellent in effectiveness and storage stability, and has a pH of 2.0 or less. More specifically, the external preparation for skin of the present invention comprises one kind or two or more kinds of organic acid(s), and one kind or two or more kinds of nonionic water soluble polymer(s) except for polysaccharides, and has a pH of 2.0 or less.

### TECHNICAL FIELD

The present invention relates to techniques for providing an external preparation for skin which is excellent in usability and storage stability irrespective of having a pH of 2.0 or less.

### BACKGROUND ART

In recent years, chemical peeling has drawn significant attention in light of its immediate consequences and effectiveness. Chemical peeling is a kind of beauty regimens for increasing metabolism of the skin by applying an external preparation for skin containing an organic acid having a particular structure to the stratum corneum on the surface of the skin, whereby allowing exfoliation of the stratum corneum. Using this regimen, amelioration of manifestations such as skin flecks, darkening, dullness, acne, acne scar, fine wrinkles and the like can be expected.

However, the external preparations for skin having a low pH for use in the chemical peeling and the like have involved problems of impossibility of uniform application due to being shed and passed on the oil content of the skin when they are applied to the skin. Further, although processes in which a degreasing agent is used prior to carrying out the chemical peeling have also known for overcoming such problems, almost no effect has been thereby achieved to improve application performances. On the other hand, attempts to formulate a variety of materials in the preparation have been made for improving the application performances, but in such cases, it is necessary to adjust the pH value of the preparation to fall within from 2 to 6 in light of the requirement to secure stability of the preparation. However, in connection with peeling agents having a pH value adjusted to fall within from 2 to 6, it has been generally known by persons attending to the chemical peeling that a more inferior peeling effect is achieved in comparison with those having a pH of 2.0 or less.

### DISCLOSURE OF THE INVENTION

The present invention was made in order to solve the foregoing problems, and an object of the invention is to provide an external preparation for skin which can be readily applied to the skin surface in a uniform manner, is excellent in effectiveness and storage stability, and is characterized by having a pH of 2.0 or less.

In order to accomplish the object described above, the present invention is directed to an external preparation for skin which comprises one kind or two or more kinds of organic acid(s) and one kind or two or more kinds of nonionic water soluble polymer(s) except for polysaccharides, and has a pH of 2.0 or less.

Preferably, the organic acid is selected from the group consisting of trichloroacetic acid, succinic acid, malic acid, glycolic acid, mandelic acid, lactic acid, α-hydroxybutyric acid, α-hydroxyvaleric acid, β-hydroxyvaleric acid, citric acid, tartronic acid, tartaric acid, glucuronic acid, gluconic acid, mucic acid, pyruvic acid, salicylic acid, ascorbic acid, catechol, and p-hydroquinone.

Preferably, the aforementioned nonionic water soluble polymer comprises a highly polymerized polyethylene glycol and/or polyvinyl alcohol.

The aforementioned external preparation for skin is useful in chemical peeling.

### BEST MODE FOR CARRYING OUT THE INVENTION

The external preparation for skin according to the present invention comprises an organic acid and a water soluble polymer.

The organic acid for use in the present invention is not particularly limited as long as it is a compound exhibiting acidity, however, it is preferably a carboxylic acid or a phenol. Moreover, specific examples the carboxylic acid include carboxylic acids such as trichloroacetic acid, succinic acid, malic acid, glycolic acid, mandelic acid, lactic acid, α-hydroxybutyric acid, α-hydroxyvaleric acid, β-hydroxyvaleric acid, citric acid, tartronic acid, tartaric acid, glucuronic acid, gluconic acid, mucic acid, pyruvic acid, salicylic acid, ascorbic acid and the like, while specific examples of the phenol include catechol, p-hydroquinone, and derivatives thereof and the like. Also, as described above, the pH of the external preparation for skin according to the present invention is set to be 2.0 or less in light of the peeling effect. When the pH is greater than 2.0, a peeling agent exhibiting excellent effect can not be obtained.

In the present invention, the amount of the organic acid as described above to be included is preferably 0.01% by weight or greater and particularly preferably 0.1% by weight or greater per total amount of the external preparation for skin. Moreover, when the organic acid is in its solid state at ordinary temperature, the amount may be the concentration of each saturated solution or less. When the included organic acid is in its liquid state at ordinary temperature, upper limit of the amount is not particularly defined.

Also, the water soluble polymer for use in the present invention may be a nonionic water soluble polymer other than polysaccharides, and a polyvinyl alcohol or a highly polymerized polyoxyethylene glycol can be suitably used. Although polyvinyl alcohols are a kind of water soluble polymers that are obtained by hydrolysis of a vinyl polyacetate, and polyvinyl alcohols having any of a variety of polymerization degrees have been placed on the market, the polymerization degree of the polyvinyl alcohol which may be used in the present invention is not particularly limited. Also, polyoxyethylene glycol for use herein is not a polyoxyethylene glycol having low polymerization degree which has been generally used as a moisturizing agent, but is preferably a polyoxyethylene glycol which has been used as a thickening agent, i.e., a highly polymerized polyoxyethylene glycol. The polymerization degree of the polyoxyethylene glycol is preferably from 1000 to 150000, and particularly preferably from 2000 to 50000.

The amount of the nonionic water soluble polymer to be included is preferably from 0.001% by weight to 20% by weight, and particularly from 0.01% by weight to 15% by weight per total amount of the external preparation for skin. The polymer may have any viscosity, and thus, any state involving liquid as well as gel is permitted.

In the external preparation for skin of the present invention may be used a lower alcohol, a polyhydric alcohol and a water soluble polymer other than the aforementioned water soluble polymers, a resin, an inorganic acid or the like in addition to the organic acid and the water soluble polymer in the range not to impair the effect of the present invention.

Site to which the external preparation for skin according to the present invention can be applied is not particularly limited, but the preparation can be used on not only the face but also all over the body such as neck, arms, waist, hip, legs and the like.

### EXAMPLE

The present invention is further explained in detail by way of Examples, however, as a matter of course, the present invention is not anyhow limited only to these Examples.

With the formulations presented in Table 1, external preparations for skin having a low pH according to Example 1 to Example 3 and Comparative Example 1 to Comparative Example 3 were prepared. The water soluble polymer used in Example 1 to Example 3 was a highly polymerized polyoxyethylene glycol (polymerization degree: 45000) or a polyvinyl alcohol, and that used in Comparative Example 1 to Comparative Example 3 was hydroxyethyl cellulose or carboxymethyl cellulose which is a polysaccharide. The carboxymethyl cellulose is an ionic water soluble polymer. Moreover, in Comparative Example 4, no water soluble polymer was included.

TABLE 1

| | Example | Example | Example | Comparative | Comparative | Comparative | Comparative |
|---|---|---|---|---|---|---|---|
| | 1 | 2 | 3 | Example 1 | Example 2 | Example 3 | Example 4 |
| Purified water | 45 | 5 | 45 | 45 | 41 | 45 | 70 |
| 70% by weight aqueous | 30 | 80 | 30 | 30 | 30 | 30 | 30 |
| solution of glycolic acid | | | | | | | |
| 2% by weight aqueous solution | 25 | 15 | — | — | — | — | — |
| of highly polymerized | | | | | | | |
| polyoxyethylene glycol | | | | | | | |
| 16% by weight aqueous solution | — | — | 25 | — | — | — | — |
| of polyvinyl alcohol | | | | | | | |
| 1% by weight hydroxyethyl | — | — | — | 25 | 25 | — | — |
| cellulose | | | | | | | |
| 1% by weight carboxymethyl | — | — | — | — | — | 25 | — |
| cellulose | | | | | | | |
| Sodium hydroxide | — | — | — | — | 4 | — | — |
| Total (% by weight) | 100 | 100 | 100 | 100 | 100 | 100 | 100 |
| pH | 1.4 | 0.5 | 1.4 | 1.4 | 3.5 | 1.4 | 1.4 |

(Process for Production)

An aqueous solution of each water soluble polymer was previously prepared according to a conventional method, and a predetermined amount was weighed. Thereto was added a 70% by weight aqueous solution of a commercially available glycolic acid, and gave 100% by weight with purified water followed by stirring to give a homogenous state. In Comparative Example 2, sodium hydroxide was added to the 70% by weight aqueous solution of glycolic acid, and the preparation was completed by adding thereto an aqueous solution of the water soluble polymer and purified water. In Comparative Example 4, preparation was carried out by diluting the aqueous solution of a commercially available glycolic acid with purified water.

(Evaluation of Application Performance and Effectiveness)

Seven flamed areas of 5 cm square were defined on forearms of ten male panels of from the twenties to the forties respectively, and then an evaluation expert applied the sample of the aforementioned Example 1 to Example 3 and Comparative Example 1 to Comparative Example 4 in the flame using a flat brush for painting. Application performance was then evaluated by the evaluation expert according to the category either of “capable of applying in a uniform manner” or “liable to be uneven”. In addition, after leaving to stand following the application for 10 minutes, the sample was washed away with water. Twenty four hours later, the evaluation expert visually determined how the stratum corneum is peeled at the part to which each sample was applied, as the evaluation of effectiveness, to categorize into any one of: “the stratum corneum being uniformly peeled”, “the stratum corneum being peeled patchwise”, “the stratum corneum not being peeled”. The results are shown in Table 2. Values in Table 2 indicate the number of the panel(s) evaluated by the evaluation expert as falling under each category.

TABLE 2

| Items of | | | | | Comparative | Comparative | Comparative | Comparative |
|---|---|---|---|---|---|---|---|---|
| evaluation | Category | Example 1 | Example 2 | Example 3 | Example 1 | Example 2 | Example 3 | Example 4 |
| Application | Capable of | 9 | 10 | 9 | 8 | 9 | 7 | 0 |
| performance | applying in a | | | | | | | |
| | uniform manner | | | | | | | |
| | Liable to be | 1 | 0 | 1 | 2 | 1 | 3 | 10 |
| | uneven | | | | | | | |
| Effectiveness | Stratum corneum | 8 | 10 | 9 | 7 | 2 | 7 | 0 |
| | being peeled | | | | | | | |
| | patchwise | | | | | | | |
| | Stratum corneum | 2 | 0 | 1 | 3 | 1 | 3 | 10 |
| | being peeled | | | | | | | |
| | patchwise | | | | | | | |
| | Stratum corneum | 0 | 0 | 0 | 0 | 7 | 0 | 0 |
| | not being peeled | | | | | | | |

From the foregoing results, it is proven that in Comparative Example 4 without including any water soluble polymer exhibited an extremely inferior application performance, and consequently, the stratum corneum on the skin surface was peeled off in a very uneven manner. To the contrary, the external preparation for skin of Example 1 to Example 3, and Comparative Example 1 to Comparative Example 3 in which the water soluble polymer was included could be readily applied to the skin in a uniform manner, and consequently, results also excellent in effectiveness could be achieved. On the other hand, when Comparative Example 2 according to the formulation having the pH adjusted to 3.5, the value beyond 2.0, is used, it was revealed that the peeling effect was extremely deteriorated although the application performance was excellent.

(Evaluation of Stability)

Extent of alteration of the viscosity at each temperature was evaluated for the samples of the aforementioned Example 1 to Example 3 and Comparative Example 1 to Comparative Example 3. The viscosity of the sample on the next day of its preparation was assumed to be 100, and the viscosity after storing at 25° C., 40° C. and 50° C. for one week is shown in Table 3 in a relative value.

TABLE 3

| | Viscosity on | 25° C./ | 40° C./ | 50° C./ |
|---|---|---|---|---|
| | the next day | 1 week | 1 week | 1 week |
| Example 1 | 100 | 93 | 96 | 83 |
| Example 2 | 100 | 91 | 81 | 72 |
| Example 3 | 100 | 91 | 83 | 65 |
| Comparative | 100 | 80 | 20 | 7 |
| Example 1 | | | | |
| Comparative | 100 | 95 | 84 | 60 |
| Example 2 | | | | |
| Comparative | 100 | 100 | 40 | 16 |
| Example 3 | | | | |

As is clear from Table 3, higher storage temperature resulted in significant tendency of decrease in viscosity in Comparative Example 1 and Comparative Example 3, and to the contrary, relative value of the viscosity following leaving to stand for one week at 50° C. is 60 or greater in Example 1 to Example 3 and Comparative Example 2, whereby revealing markedly excellent storage stability in comparison with Comparative Example 1 and Comparative Example 3.

Further, upon successive measurement of the viscosity for the sample of Example 1 to Example 3, tendency of additional decrease in viscosity was not observed after elapse of one week.

The results hereinabove are summarized in Table 4 below.

TABLE 4

| | | | | Comparative | Comparative | Comparative | Comparative |
|---|---|---|---|---|---|---|---|
| | Example 1 | Example 2 | Example 3 | Example 1 | Example 2 | Example 3 | Example 4 |
| Application | Uniform | Uniform | Uniform | Uniform | Uniform | Uniform | Unevenness |
| performance | | | | | | | |
| Effectiveness | Favorable | Favorable | Favorable | Favorable | No effect | Favorable | patchy |
| Storage | Favorable | Favorable | Favorable | Unfavorable | Favorable | Unfavorable | — |
| stability | | | | | | | |
| Total | ◯ | ◯ | ◯ | X | X | X | X |
| assessment | | | | | | | |

Accordingly, it was elucidated that excellent usability and peeling effect, as well as securement of stability which is satisfactory as a product could be achieved according to the cases in which formulations in Example 1 to Example 3 were employed, and that highly polymerized polyoxyethylene glycols and polyvinyl alcohols which are not classified in polysaccharides and ionic water soluble polymers are suitable as the water soluble polymer.

As explained in detail hereinabove, an external preparation for skin which is excellent in usability and storage stability irrespective of having a low pH, and which is particularly suitable as a peeling agent can be provided according to the present invention.

### INDUSTRIAL APPLICABILITY

The external preparation for skin of the present invention is useful as a chemical peeling agent.

1. An external preparation for skin which comprises one kind or two or more kinds of organic acid(s) and one kind or two or more kinds of nonionic water soluble polymer(s) except for polysaccharides, and is characterized by having a pH of 2.0 or less.

2. (canceled)

3. (canceled)

4. (canceled)

5. The external preparation for skin according to claim 1 wherein the preparation has a pH of 1.4 or less.

6. An external preparation for skin which comprises glycolic acid and polyvinyl alcohol, and has a pH of 2.0 or less.

7. The external preparation for skin according to claim 6 wherein the preparation has a pH of 1.4 or less.

8. A method of chemically peeling skin comprising contacting the skin with the preparation of claim 1.

9. A method of chemically peeling skin comprising contacting the skin with the preparation of claim 5.

10. A method of chemically peeling skin comprising contacting the skin with the preparation of claim 6.

11. A method of chemically peeling skin comprising contacting the skin with the preparation of claim 7.
